(19) United States
(12) Patent Application Publication
(10) Pub. No.: US 2024/0207548 A1
(43) Pub. Date: Jun. 27, 2024

(54) BREATHING MACHINE AND TURBO BLOWER SUBASSEMBLY THEREOF

(71) Applicant: RESVENT MEDICAL TECHNOLOGY CO., LTD, Shenzhen (CN)

(72) Inventors: Jiawei Huang, Shenzhen (CN); Feicun Fang, Shenzhen (CN); Wengui Zhang, Shenzhen (CN)

(73) Assignee: RESVENT MEDICAL TECHNOLOGY CO., LTD, Shenzhen (CN)

(21) Appl. No.: 18/230,867

(22) Filed: Aug. 7, 2023

(30) Foreign Application Priority Data
Dec. 27, 2022 (CN) 202211685657.7

(51) Int. Cl. A61M 16/00

(52) U.S. Cl. CPC A61M 16/0066; A61M 16/0003

(57) ABSTRACT

A turbo blower subassembly is provided with a shell and a turbo blower. The shell includes a main body, an air inlet cover, a sealing cover, and an accommodating cavity. The main body has a side wall and a bottom wall, the sealing cover covers an end of the main body. The accommodating cavity is enclosed by the side wall, the bottom wall and the sealing cover. The air inlet cover covers an end of the main body. an air inlet channel in communication with the accommodating cavity is formed between the air inlet cover and the bottom wall, the air inlet cover is provided with an air inlet port in communication with the air inlet channel. The turbo blower is accommodated in the accommodating cavity, and fixedly mounted between the main body and the sealing cover. There is a breath machine is also provided.

### CROSS REFERENCE TO RELATED APPLICATION

This non-provisional patent application claims priority under 35 U. S. C. § 119 from Chinese Patent Application No. 2022116856577 filed on Dec. 27, 2022, the entire content of which is incorporated herein by reference.

### TECHNICAL FIELD

The disclosure relates to biology technologies, in particular to serum metabolic biomarkers and a kit for detecting drug-resistant tuberculosis.

### BACKGROUND

With the increasing development of medical technology, more and more people are aware of the hazards of respiratory diseases. As the medical equipment for treating certain respiratory diseases, breathing machines requires high reliability, in which the turbine is the most crucial component used for generating airflow. The turbine box is a carrying component for the turbine, and plays various roles such as mixing gas, stabilizing airflow, reducing noise, silencing, isolating vibration, conducting heat, and cooling. However, the turbine generates noise during operation, which may have an impact on the users of breathing machines. At present, mainstream solutions mainly include using a sponge to completely fill the inner cavity of the turbine box, or using a sponge to cover the primary noise source such as the air inlet/outlet outside the turbine box, and absorbing noise through the microporous structure of the sponge to reduce noise. The prior art mainly relies on the texture of the sponge to achieve noise reduction, but are limited by the texture in turn.

Therefore, there is a critical need to improve the detection and diagnosis of drug-resistant tuberculosis.

### SUMMARY

There is provided a breathing machine and a turbo blower subassembly, that are capable of reducing noise generated by a turbo blower contained in the breathing machine or the turbo blower.

In a first aspect, a turbo blower subassembly is provided with a shell and a turbo blower. The shell has a main body, an air inlet cover, a sealing cover, and an accommodating cavity. The main body has a side wall and a bottom wall. The sealing cover covers an end of the main body away from the bottom wall. The accommodating cavity is enclosed by the side wall, the bottom wall and the sealing cover. The air inlet cover covers an end of the main body closed to the bottom wall, an air inlet channel in communication with the accommodating cavity being formed between the air inlet cover and the bottom wall. The air inlet cover is provided with an air inlet port in communication with the air inlet channel. The turbo blower is accommodated in the accommodating cavity, and fixedly mounted between the main body and the sealing cover.

In a second aspect, a breathing machine is provided with a housing and a turbo blower subassembly. The housing is provided with a mounting cavity. The turbo blower subassembly is arranged in the mounting cavity, and the turbo blower subassembly includes a shell and a turbo blower. The shell has a main body, an air inlet cover, a sealing cover, and an accommodating cavity. The main body has a side wall and a bottom wall. The sealing cover covers an end of the main body away from the bottom wall. The accommodating cavity is enclosed by the side wall, the bottom wall and the sealing cover. The air inlet cover covers an end of the main body closed to the bottom wall, an air inlet channel in communication with the accommodating cavity being formed between the air inlet cover and the bottom wall. The air inlet cover is provided with an air inlet port in communication with the air inlet channel. The turbo blower is accommodated in the accommodating cavity, and fixedly mounted between the main body and the sealing cover.

In a second aspect, a turbo blower subassembly is provided with a shell and a turbo blower. The shell has a main body, an air inlet cover, a sealing cover, and an accommodating cavity. The sealing cover and the air inlet cover respectively covers opposite sides of the main body. The accommodating cavity is enclosed by the main body and the sealing cover. The air inlet channel in communication with the accommodating cavity is formed between the air inlet cover and main body, the air inlet cover being provided with an air inlet port in communication with the air inlet channel. The turbo blower is accommodated in the accommodating cavity, and fixedly mounted between the main body and the sealing cover.

In the foregoing embodiment, the purposes of sound insulation, silencing, sound conduction, and noise reduction are achieved through the overall structural design of the shell of the turbofan subassembly, rather than adopting acoustic foam. The air inlet groove and the air inlet channel are not only air channels, but also can be used for noise reduction, thereby optimizing the noise reduction effect and reducing the overall size of the shell. The design of circular radial stacked shell can form a plurality of cavities and walls for physical silencing, so that the overall air channels are designed as air paths where the air is concentrated, divergent, and then concentrated. As a carrying component of the turbo blower, the shell can effectively reduce the quantity of parts in the overall turbo blower subassembly, and greatly reduce assembly and material costs. It also has the effects of mixing airflow, stabilizing airflow, reducing noise, silencing, isolating vibration, conducting heat, cooling, and the like, and greatly improves the stability of the turbo blower subassembly and user experience.

### BRIEF DESCRIPTION OF THE DRAWINGS

In order to illustrate the technical solution in the embodiments of the disclosure or the prior art more clearly, a brief description of drawings required in the embodiments or the prior art is given below. Obviously, the drawings described below are only some of the embodiments of the disclosure. For ordinary technicians in this field, other drawings can be obtained according to the structures shown in these drawings without any creative effort.

FIG. 1 illustrates an isometric diagram of a turbo blower subassembly in accordance with an embodiment.

FIG. 2 illustrates a first exploded schematic diagram of a turbo blower subassembly in accordance with an embodiment.

FIG. 3 illustrates a second exploded schematic diagram of a turbo blower subassembly in accordance with an embodiment.

FIG. 4 illustrates a first section diagram of a turbo blower subassembly in accordance with an embodiment.

FIG. 5 illustrates a second section diagram of a turbo blower subassembly in accordance with an embodiment.

FIG. 6 illustrates an isometric diagram of a main body of a turbo blower subassembly shown in FIG. 1;

FIG. 7 illustrates a cross-sectional schematic diagram of a main body of a turbo blower subassembly shown in FIG. 1; and

FIG. 8 illustrates a schematic diagram of a breathing machine in accordance with an embodiment.

### DETAILED DESCRIPTION OF THE EMBODIMENTS

In the description of the present application, it should be understood that the orientations or positional relationships indicated by the terms “length”, “width”, “upper”, “lower”, “front”, “back”, “left”, “right”, “vertical”, “horizontal”, “top”, “bottom”, “inner”, “outer”, and the like are based on the orientations or positional relationships shown in the accompanying drawings, which are merely intended to facilitate and simplify the description of the present application only, but not to indicate or imply that the device or element referred to must have a particular orientation or be constructed and operated in a particular orientation, and therefore, cannot be interpreted as limiting the present application.

In addition, the terms “first” and “second” are merely for the sake of description, and cannot be understood as indicating or implying the relative importance or implicitly indicating the quantity of the indicated technical features. Therefore, the features defined by the terms “first” and “second” may explicitly or implicitly include one or more of these features. In the description of the present application, the term “plurality” refers to two or more, unless otherwise specifically defined.

In the description of the present application, unless otherwise explicitly specified and defined, the terms “mounted”, “coupled”, “connected”, “fixed”, and the like should be construed broadly, for example, the term “connected” may be fixedly connected, detachably connected, or integrated connected; mechanically or electrically connected; directly or indirectly connected through a medium, intercommunication between two elements, or interaction between two elements. Those of ordinary skill in the art may understand the specific meanings of the above terms in the present application according to specific circumstances.

In order to understand the content of the present application more clearly and accurately, detailed descriptions will be provided with reference to the accompanying drawings. The drawings in the specification show examples of embodiments of the present application, where the same reference numerals represent the same elements. It should be understood that the proportions shown in the drawings of the specification are not actual proportions implemented in the present application, which are merely for illustrative purposes, and are not drawn according to original dimensions.

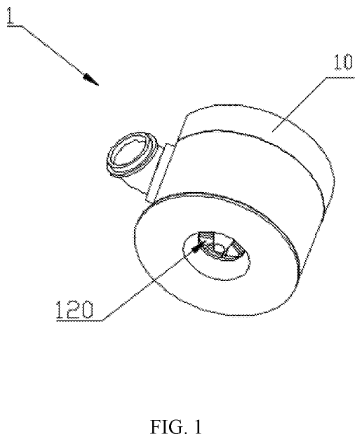

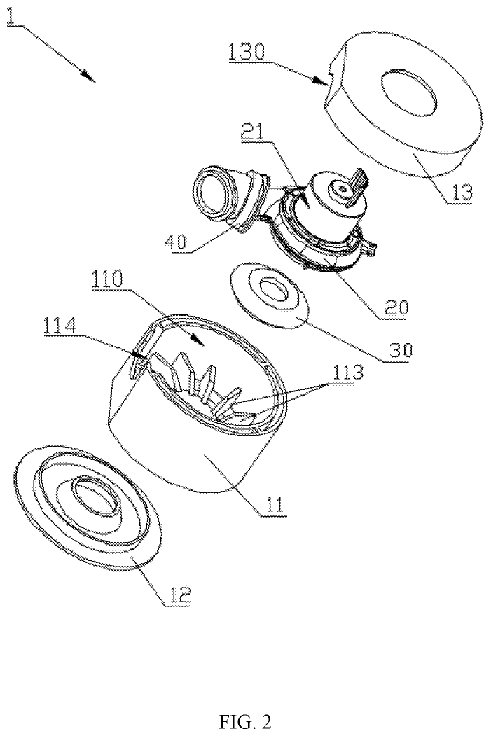

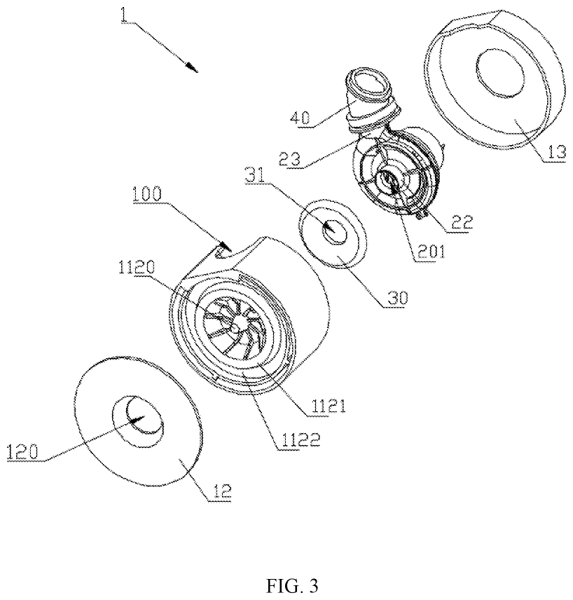

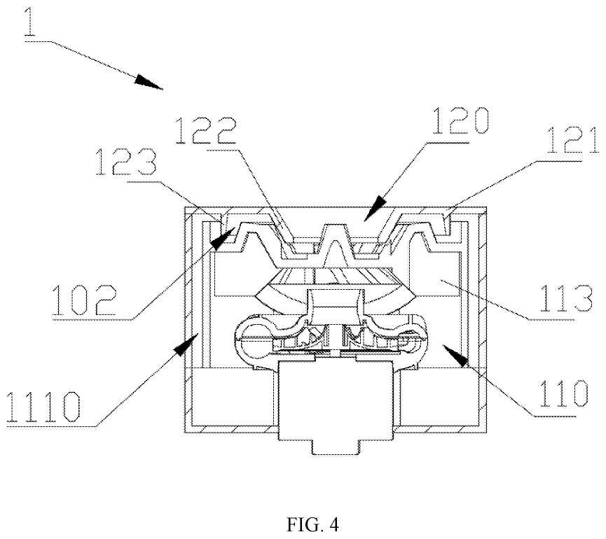

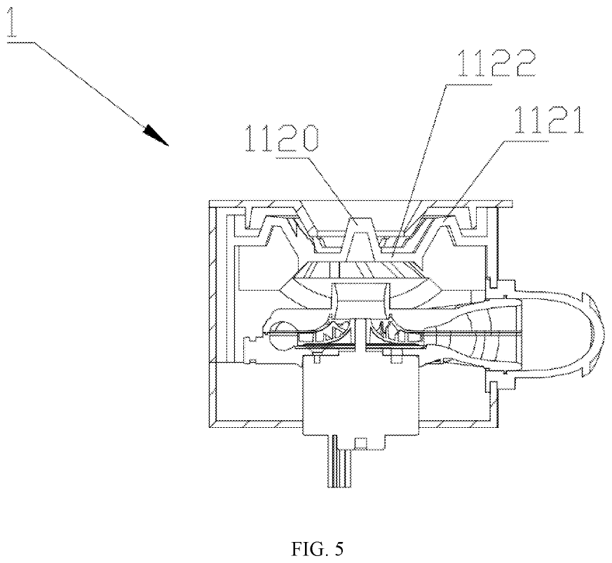

Referring to FIG. 1 to FIG. 5, FIG. 1 is a three-dimensional diagram of a turbo blower subassembly provided in an embodiment of the present application, FIG. 2 is a first exploded schematic diagram of a turbo blower subassembly provided in an embodiment of the present application, FIG. 3 is a second exploded schematic diagram of a turbo blower subassembly provided in an embodiment of the present application, FIG. 4 is a first section diagram of a turbo blower subassembly provided in an embodiment of the present application, and FIG. 5 is a second section diagram of a turbo blower subassembly provided in an embodiment of the present application. A turbo blower subassembly 1 is applied to ventilation therapy equipment such as breathing machines, and is the most core device of the ventilation therapy equipment. In some feasible embodiments, the turbo blower subassembly 1 may also be applied to equipment provided with a turbo blower, such as a vacuum cleaner or an air blower, without limitation here.

The turbo blower subassembly 1 includes a shell 10 and a turbo blower 20. The shell 10 is provided with an accommodating cavity 110, and the turbo blower 20 is accommodated in the accommodating cavity 110.

The shell 10 includes a main body 11, an air inlet cover 12, and a sealing cover 13. In this embodiment, the main body 11 includes a side wall 111 and a bottom wall 112, and the bottom wall 112 is connected to one end of the side wall 111. Specifically, the side wall 111 includes a first side wall 1111 and a second side wall 1112, the bottom wall 112 is connected to one end of the first side wall 1111, and the second side wall 1112 is sheathed on an outer side of the first side wall 1111. An air inlet groove 1110 is formed between the first side wall 1111 and the second side wall 1112. It should be understood that the main body 11 has double solid walls.

In this embodiment, the side wall 111 is tubular, and the entire bottom wall 112 is circular. Specifically, both the first side wall 1111 and the second side wall 1112 are approximately hollow and tubular. Correspondingly, the air inlet groove 1110 between the first side wall 1111 and the second side wall 1112 is approximately circular. In some feasible embodiments, an exterior of the side wall 111 may be square or irregular, and an interior of the side wall 111 is circular. That is, the second side wall 1112 may be square or irregular, and the first side wall 1111 is hollow and tubular regardless of the shape of the second side wall 1112.

The sealing cover 13 covers an end, away from the bottom wall 112, of the main body 11, and the air inlet cover 12 covers an end, close to the bottom wall 112, of the main body 11. The accommodating cavity 110 is enclosed by the side wall 111, the bottom wall 112, and the sealing cover 13. Specifically, the sealing cover 13 covers an end, away from the bottom wall 112, of the second side wall 1112, the air inlet cover 12 covers an end, close to the bottom wall 112, of the second side wall 1112, and the accommodating cavity 110 is enclosed by the first side wall 1111, the bottom wall 112, and the sealing cover 13. The second side wall 1112 is sheathed on a side, away from the accommodating cavity 110, of the first side wall 1111. In this embodiment, an end, away from the bottom wall 112, of the first side wall 1111 is flush with the end, away from the bottom wall 112, of the second side wall 1112, and the first side wall 1111 and the second side wall 1112 are fixedly connected through a fixing portion 1113.

The air inlet cover 12 covers a side, away from the accommodating cavity 110, of the bottom wall 112, and an air inlet channel 102 in communication with the accommodating cavity 110 is formed between the air inlet cover 12 and the bottom wall 112. The air inlet cover 12 is provided with an air inlet port 120 in communication with the air inlet channel 102. The air inlet groove 1110 is in communication with the air inlet channel 102 and the accommodating cavity 110. It should be understood that the air inlet port 120, the air inlet channel 102, the air inlet groove 1110, and the accommodating cavity 110 are sequentially in communication with each other. In this embodiment, the entire air inlet cover 12 is circular, and the air inlet port 120 is positioned in the center of the air inlet cover 12.

When the turbo blower 20 accommodated in the accommodating cavity 110 operates, the airflow needs to enter the shell 10 from the air inlet port 120 and passes through the air inlet channel 102 and the air inlet groove 1110 sequentially before reaching the accommodating cavity 110. The air inlet groove 1110 between the first side wall 1111 and the second side wall 1112 is approximately circular, and the cross-sectional area of the air inlet groove 1110 increases with the radius of the side wall 111.

Further, when the turbo blower 20 accommodated in the accommodating cavity 110 operates, the noise generated by the turbo blower 20 can be propagated to the outside of the shell 10 through an air passage after passing through the accommodating cavity 110, the air inlet groove 1110, the air inlet channel 102, and the air inlet port 120 sequentially. The configuration and coordination of the main body 11 with the air inlet cover 12 and the sealing cover 13 effectively increase the passing distance of noise, which can greatly reduce noise to achieve the purpose of multiple noise reduction.

The air inlet cover 12 includes a cover 121 and an air inlet wall 122, one end of the air inlet wall 122 is connected to the cover 121, and the other end of the air inlet wall 122 extends towards the bottom wall 112. In this embodiment, the size of the air inlet wall 122 at the end close to the bottom wall 112 is smaller than that of the air inlet wall 122 at the other end away from the bottom wall 112. The air inlet port 120 is enclosed by the air inlet wall 122. Correspondingly, the cover 121 is circular; and the air inlet wall 122 is arranged in the center of the cover 121.

In this embodiment, a side, facing the bottom wall 112, of the cover 121 is provided with raised ribs 123, and the raised ribs 123 extend from one side of the cover 121 towards the bottom wall 112. Specifically, the raised ribs 123 are arranged in a ring shape on the periphery of the air inlet wall 122.

The bottom wall 112 includes a plurality of protrusions 1121 and recessions 1122. The recessions 1122 are recessed away from the air inlet cover 12 from the bottom wall 112, and are arranged opposite to the air inlet wall 122 and the raised ribs 123; and the protrusions 1121 protrude from the bottom wall 112 towards the air inlet cover 12, and are arranged opposite to the air inlet port 120 or arranged between the air inlet wall 122 and the raised ribs 123. In this embodiment, the recessions 1122 and the protrusions 1121 are alternately arranged. Some recessions 1122 are arranged opposite to the air inlet wall 122, and other recessions 1122 are arranged opposite to the raised ribs 123. One of the protrusions 1121 is arranged opposite to the air inlet port 120, and other protrusions 1121 are arranged between the air inlet wall 122 and the raised ribs 123. It should be understood that the protrusions 1121 and recessions 1122 of the bottom wall 112 are mutually matched and alternately arranged with the air inlet wall 122 and raised ribs 123 of the air inlet cover 12, so that the entire air inlet channel 102 is tortuous S-shaped in the cross-sectional view, which can increase the length of the air inlet channel 102. Meanwhile, the tortuous S-shaped air inlet channel 102 can also form a radial overlap of channel wall projections, and the thickness of the overlap is 5 times or more than the conventional wall thickness, which can effectively reduce noise and achieve a better sound insulation effect.

In this embodiment, the protrusions 1121 includes a boss 1120 opposite to the air inlet port 120. Specifically, an end, away from the accommodating cavity 110, of the boss 1120 extends from an end, close to the bottom wall 112, of the air inlet wall 122 to the air inlet port 120. It should be understood that the end, away from the accommodating cavity 110, of the boss 1120 is further away from the bottom wall 112 than the end, close to the accommodating cavity 110, of the air inlet wall 122.

An outer wall of the boss 1120 is inclined, and the size of the end, close to the accommodating cavity 110, of the boss 1120 is greater than that of the end, away from the accommodating cavity 110, of the boss 1120. That is, the entire boss 1120 is in a trapezoidal cylindrical shape, namely, a truncated cone. The design of the boss 1120 can effectively weaken the stiffness of the bottom wall 112 in an axial direction, and guide most noises generated by the turbo blower 20 to the boss 1120 for divergence through resonant propagation. Meanwhile, the boss 1120 makes the air inlet channel circular tiled from a solid circle, which can effectively disperse and evenly distribute the concentrated airflow entering from the air inlet port 120 to the entire air inlet channel 102 to form multiple dispersed airflow streams, so that the flow rate of the dispersed airflow can be effectively slowed down, and the wind noise of the airflow can be reduced. In some feasible embodiments, the boss 1120 may alternatively be hemispherical, ellipsoidal, conical, or the like, which is not limited here. In other feasible embodiments, the entire boss 1120 may alternatively be a recession which extends away from the air inlet port 120 from the bottom wall 112.

The shell 10 is provided with a limiting hole 100. In this embodiment, the sealing cover 13 is provided with a notch 130, the main body 11 is correspondingly provided with a through hole 114, and the notch 130 and the through hole 114 jointly form the limiting hole 100. Specifically, the through hole 114 is formed at an end, close to the sealing cover 13, of the side wall 111.

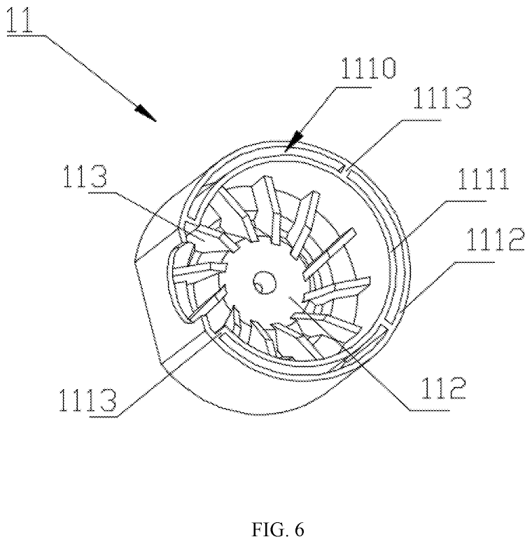

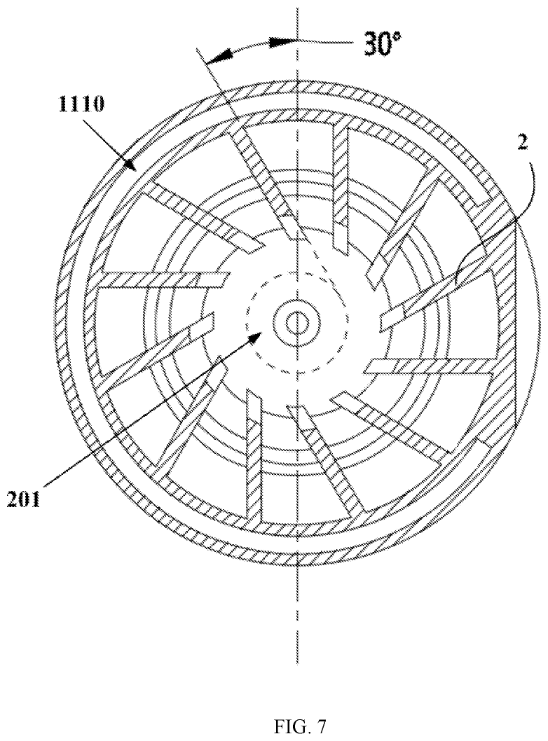

Referring to FIG. 6 and FIG. 7, FIG. 6 is a three-dimensional diagram of a main body provided in an embodiment of the present application, and FIG. 7 is a cross-sectional schematic diagram of a main body provided in an embodiment of the present application. In this embodiment, a plurality of reinforcing ribs 113 are arranged on a side, facing the turbo blower 20, of the bottom wall 112. The reinforcing ribs 113 are arranged at intervals in the circumferential direction of the bottom wall 112, and arranged on the bottom wall 112 in a preset direction. Specifically, each reinforcing rib 113 is in a sheet shape and connected to a side, facing the accommodating cavity 110, of the bottom wall 112 and a side, facing the accommodating cavity 110, of the first side wall 1111. The reinforcing ribs 113 can increase the stiffness of the connection between the bottom wall 112 and the first side wall 1111, reduce the occurrence of the resonance between the bottom wall 112 and the first side wall 1111, and reduce the propagation of noise accordingly.

In this embodiment, the turbo blower 20 is fixedly mounted between the main body 11 and the sealing cover 13. Specifically, the turbo blower 20 is fixedly mounted between the bottom wall 112 and the sealing cover 13. The turbo blower 20 includes fan blades 21 and an air inlet pipe 22, the air inlet pipe 22 is arranged on a side, facing the bottom wall 112, of the turbo blower 20, and an air inlet port 201 is arranged on the air inlet pipe 22. The air inlet pipe 22 is tubular, and the air inlet port 201 is arranged opposite to the boss 1120.

The fan blades 21 include a rotation direction, and the circumferential direction of the reinforcing ribs 113 along the bottom wall 112 is the same as the rotation direction. That is, if the rotation direction of the fan blades 21 is clockwise, the reinforcing ribs 113 are sequentially arranged clockwise; or if the rotation direction of the fan blades 21 is counterclockwise, the reinforcing ribs 113 are sequentially arranged counterclockwise. There is a preset angle between the preset direction of the reinforcing ribs 113 and the diameter of the fan blades 21, and a straight line in the preset direction is tangent to a wall of the air inlet pipe 22. In this embodiment, the preset angle is 30°. In some feasible embodiments, the preset angle may alternatively be 20°, 25°, 35°, 40°, 45° or other degrees, which is not limited here.

The preset angle between the preset direction of the reinforcing ribs 113 and the diameter of the fan blades 21 is 30°, and the preset direction is tangent to the wall of the air inlet pipe 22, so that the reinforcing ribs 113 can guide an air flow direction, the air flow direction from the air inlet port 201 into the turbo blower 20 is identical with and tangent to the rotation direction of the fan blades 21, the amplitude of turbulence generated by the collision of the airflow with the fan blades 21 is reduced accordingly, and noise reduction and silencing are ultimately achieved.

Referring to FIG. 2 again, the turbo blower 20 further includes an air outlet pipe 23, and the air outlet pipe 23 protrudes from the limiting hole 100 to the outside of the shell 10. The turbo blower subassembly 1 further includes a sealing element 40, and the sealing element 40 is sheathed at an end, away from the accommodating cavity 110, of the air outlet pipe 23 and sealed between the air outlet pipe 23 and the shell 10.

The turbo blower subassembly 1 further includes a shock absorber 30, and the shock absorber 30 is arranged between the turbo blower 20 and the reinforcing ribs 113. It should be understood that the shock absorber 30 is fixed to ends, away from the bottom wall 112, of the reinforcing ribs 113, and the turbo blower 20 is fixedly arranged between the shock absorber 30 and the sealing cover 13. In this embodiment, the shock absorber 30 is provided with a through hole 31, and the air inlet pipe 22 is accommodated in the through hole 31. The shock absorber 30 is a disc spring part. When the turbo blower 20 operates, the air pressure of airflow at the air inlet port 201 will fluctuate, causing the entire turbo blower 20 to vibrate up and down with the rotation of the fan blades 21 to generate noise. The shock absorber 30 is arranged at the air inlet port 201 of the turbo blower 20 to support the turbo blower 20. When the turbo blower 20 vibrates up and down, the shock absorber 30 can correspondingly generate deformation and damping to provide stable vibration damping, so as to reduce the amplitude of the turbo blower 20 and achieve noise reduction. In this embodiment, the shock absorber 30 is made of silicone. In some feasible embodiments, the shock absorber 30 may alternatively be made of a non-rigid material, including but not limited to soft materials such as Thermoplastic Urethane (TPU) and Thermoplastic Elastomer (TPE).

In the foregoing embodiment, the purposes of sound insulation, silencing, sound conduction, and noise reduction are achieved through the overall structural design of the shell of the turbofan subassembly, rather than adopting acoustic foam. The air inlet groove and the air inlet channel are not only air channels, but also can be used for noise reduction, thereby optimizing the noise reduction effect and reducing the overall size of the shell. The design of circular radial stacked shell can form a plurality of cavities and walls for physical silencing, so that the overall air channels are designed as air paths where the air is concentrated, divergent, and then concentrated. As a carrying component of the turbo blower, the shell can effectively reduce the quantity of parts in the overall turbo blower subassembly, and greatly reduce assembly and material costs. It also has the effects of mixing airflow, stabilizing airflow, reducing noise, silencing, isolating vibration, conducting heat, cooling, and the like, and greatly improves the stability of the turbo blower subassembly and user experience.

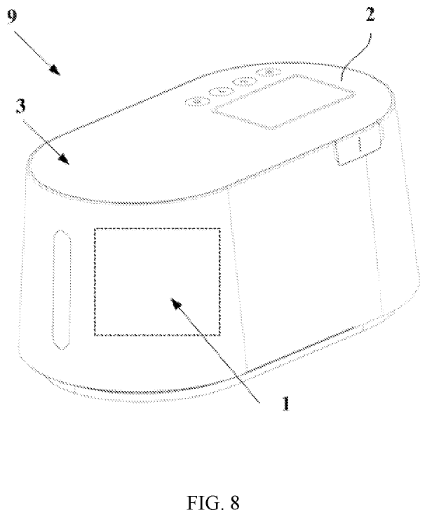

FIG. 8 is a schematic diagram of a breathing machine provided in an embodiment of the present application. A breathing machine 9 includes a housing 2 and a turbo blower subassembly 1. A specific structure of the turbo blower subassembly 1 refers to the foregoing embodiment.

In this embodiment, the housing 2 is provided with a mounting cavity 3, and the turbo blower subassembly 1 is arranged in the mounting cavity 3.

In the above embodiment, the breathing machine 9 employs all the technical solutions of all the foregoing embodiments, and therefore has at least all the beneficial effects brought by the technical solutions of the foregoing embodiments, which will not be repeated here.

Apparently, those skilled in the art can make various modifications and variations to the present application without departing from the spirit and scope of the present application. Provided that these modifications and variations of the present application fall into the scope of the claims of the present application and equivalent technologies thereof, the present application is intended to include these modifications and variations.

What are listed above are merely preferred embodiments of the present application, and are certainly not intended to limit the scope of the claims of the present application. Therefore, equivalent variations made in accordance with the claims of the present application still fall within the scope of the present application.

1. A turbo blower subassembly, comprising:
a shell, having a main body, an air inlet cover, a sealing cover, and an accommodating cavity, the main body having a side wall and a bottom wall, the sealing cover covering an end of the main body away from the bottom wall; the accommodating cavity being enclosed by the side wall, the bottom wall and the sealing cover; the air inlet cover covering an end of the main body closed to the bottom wall, an air inlet channel in communication with the accommodating cavity being formed between the air inlet cover and the bottom wall, the air inlet cover being provided with an air inlet port in communication with the air inlet channel; and
a turbo blower, being accommodated in the accommodating cavity, and being fixedly mounted between the main body and the sealing cover.

2. The turbo blower subassembly of claim 1, wherein the side wall comprising a first side wall and a second side wall, the accommodating cavity is enclosed by the first side wall, the bottom wall, and the sealing cover; the second side wall is sheathed on an outer side of the first side wall away from the accommodating cavity; an air inlet groove is formed between the first side wall and the second side wall.

3. The turbo blower subassembly of claim 2, wherein the air inlet cover comprises a cover and an air inlet wall, one end of the air inlet wall is connected to the cover, and the other end of the air inlet wall extends towards the bottom wall; the air inlet port is enclosed by the air inlet wall; a side of the cover facing to the bottom wall is provided with raised ribs, the raised ribs extend from one side of the cover towards the bottom wall; the bottom wall comprises a plurality of protrusions and recessions, the recessions are recessed away from the air inlet cover from the bottom wall, and are arranged opposite to the air inlet wall and the raised ribs; and the protrusions protrude from the bottom wall towards the air inlet cover, and are arranged opposite to the air inlet port or arranged between the air inlet wall and the raised ribs.

4. The turbo blower subassembly of claim 3, wherein the size of the air inlet wall at the end close to the bottom wall is smaller than that of the air inlet wall at the other end away from the bottom wall, the protrusions comprises a boss opposite to the air inlet port; an end, away from the accommodating cavity, of the boss extends from an end, close to the bottom wall, of the air inlet wall to the air inlet port.

5. The turbo blower subassembly of claim 4, wherein the side wall is tubular, and the entire bottom wall is circular, a plurality of reinforcing ribs are arranged on a side, facing the turbo blower, the reinforcing ribs are arranged at intervals in the circumferential direction of the bottom wall.

6. The turbo blower subassembly of claim 5, wherein the turbo blower includes fan blades, the fan blades has a rotation direction, there is a preset angle between the preset direction of the reinforcing ribs and the diameter of the fan blades, the circumferential direction of the reinforcing ribs along the bottom wall is the same as the rotation direction.

7. The turbo blower subassembly of claim 6, wherein the turbo blower subassembly further includes a shock absorber, the shock absorber is arranged between the turbo blower and the reinforcing ribs; a side of the turbo blower facing to the bottom wall defines an air inlet port, the turbo blower further includes an air outlet pipe, the air inlet port is arranged on the air inlet pipe, the shock absorber is provided with a through hole, the air inlet pipe is accommodated in the through hole.

8. The turbo blower subassembly of claim 7, wherein the air inlet port is arranged opposite to the boss, the air inlet pipe is tubular, the preset direction is tangent to the wall of the air inlet pipe, the preset angle is 30°.

9. The turbo blower subassembly of claim 1, wherein the turbo blower further includes an air outlet pipe, the air outlet pipe protrudes from the limiting hole to the outside of the shell, the turbo blower subassembly further includes a sealing element, and the sealing element is sheathed at an end, away from the accommodating cavity, of the air outlet pipe and sealed between the air outlet pipe and the shell.

10. A breathing machine, wherein, the breathing machine comprising:
a housing, provided with a mounting cavity; and
a turbo blower subassembly, arranged in the mounting cavity, and the turbo blower subassembly comprising:
a shell, having a main body, an air inlet cover, a sealing cover, and an accommodating cavity, the main body having a side wall and a bottom wall, the sealing cover covering an end of the main body away from the bottom wall; the accommodating cavity being enclosed by the side wall, the bottom wall and the sealing cover; the air inlet cover covering an end of the main body closed to the bottom wall, an air inlet channel in communication with the accommodating cavity being formed between the air inlet cover and the bottom wall, the air inlet cover being provided with an air inlet port in communication with the air inlet channel; and
a turbo blower, being accommodated in the accommodating cavity, and being fixedly mounted between the main body and the sealing cover.

11. The breathing machine of claim 10, wherein the side wall comprising a first side wall and a second side wall, the accommodating cavity is enclosed by the first side wall, the bottom wall, and the sealing cover; the second side wall is sheathed on an outer side of the first side wall away from the accommodating cavity; an air inlet groove is formed between the first side wall and the second side wall.

12. The breathing machine of claim 11, wherein the air inlet cover comprises a cover and an air inlet wall, one end of the air inlet wall is connected to the cover, and the other end of the air inlet wall extends towards the bottom wall; the air inlet port is enclosed by the air inlet wall; a side of the cover facing to the bottom wall is provided with raised ribs, the raised ribs extend from one side of the cover towards the bottom wall; the bottom wall comprises a plurality of protrusions and recessions, the recessions are recessed away from the air inlet cover from the bottom wall, and are arranged opposite to the air inlet wall and the raised ribs; and the protrusions protrude from the bottom wall towards the air inlet cover, and are arranged opposite to the air inlet port or arranged between the air inlet wall and the raised ribs.

13. The breathing machine of claim 12, wherein the size of the air inlet wall at the end close to the bottom wall is smaller than that of the air inlet wall at the other end away from the bottom wall, the protrusions comprises a boss opposite to the air inlet port; an end, away from the accommodating cavity, of the boss extends from an end, close to the bottom wall, of the air inlet wall to the air inlet port.

14. The breathing machine of claim 13, wherein the side wall is tubular, and the entire bottom wall is circular, a plurality of reinforcing ribs are arranged on a side, facing the turbo blower, the reinforcing ribs are arranged at intervals in the circumferential direction of the bottom wall.

15. The breathing machine of claim 14, wherein the turbo blower includes fan blades, the fan blades has a rotation direction, there is a preset angle between the preset direction of the reinforcing ribs and the diameter of the fan blades, the circumferential direction of the reinforcing ribs along the bottom wall is the same as the rotation direction.

16. The breathing machine of claim 15, wherein the turbo blower subassembly further includes a shock absorber, the shock absorber is arranged between the turbo blower and the reinforcing ribs; a side of the turbo blower facing to the bottom wall defines an air inlet port, the turbo blower further includes an air outlet pipe, the air inlet port is arranged on the air inlet pipe, the shock absorber is provided with a through hole, the air inlet pipe is accommodated in the through hole.

17. The breathing machine of claim 16, wherein the air inlet port is arranged opposite to the boss, the air inlet pipe is tubular, the preset direction is tangent to the wall of the air inlet pipe, the preset angle is 30°.

18. The breathing machine of claim 10, wherein the turbo blower further includes an air outlet pipe, the air outlet pipe protrudes from the limiting hole to the outside of the shell, the turbo blower subassembly further includes a sealing element, and the sealing element is sheathed at an end, away from the accommodating cavity, of the air outlet pipe and sealed between the air outlet pipe and the shell.

19. A turbo blower subassembly, comprising:
a shell, having a main body, an air inlet cover, a sealing cover, and an accommodating cavity, the sealing cover and the air inlet cover respectively covering opposite sides of the main body, the accommodating cavity being enclosed by the main body and the sealing cover; an air inlet channel in communication with the accommodating cavity being formed between the air inlet cover and main body, the air inlet cover being provided with an air inlet port in communication with the air inlet channel; and
a turbo blower, being accommodated in the accommodating cavity, and being fixedly mounted between the main body and the sealing cover.

20. The turbo blower subassembly of claim 19, wherein the main body has a bottom wall and a side wall extend from the bottom wall, the sealing cover covers an end of the main body away from the bottom wall; the air inlet cover covers an end of the main body closed to the bottom wall, the side wall comprises a first side wall and a second side wall, the accommodating cavity is enclosed by the first side wall, the bottom wall, and the sealing cover; the second side wall is sheathed on an outer side of the first side wall away from the accommodating cavity; an air inlet groove is formed between the first side wall and the second side wall.
